(19) United States
(12) Patent Application Publication
(10) Pub. No.: US 2003/0171801 A1
(43) Pub. Date: Sep. 11, 2003

(54) Partially covered intraluminal support device

(76) Inventor: Brian Bates, Bloomington, IN (US)

Correspondence Address: J. Matthew Buchanan, BRINKS HOFER GILSON & LIONE, P.O. Box 10395, Chicago, IL, 60610, US

(21) Appl. No.: 10/092,095

(22) Filed: Mar. 6, 2002

(51) Int. Cl. A61F 2/06

(52) U.S. Cl. 623/1.13

(57) ABSTRACT

An intraluminal support device for providing artificial support to a body vessel is provided. The support device includes a support frame having a substantially uniform circumference along its length, and a graft material disposed on a portion of the support frame. The graft material spans at least a portion of the length of the support frame and extends only a partial distance along the circumference of the support frame. Radiopaque markers can be disposed on the support frame adjacent the graft material to facilitate orientating the device in a body vessel such that the graft material is in a desired position relative to a treatment site of the vessel. A delivery apparatus for introducing such an intraluminal support device to a vessel is also provided.

### FIELD OF THE INVENTION

The present invention relates generally to the field of intraluminal support devices, or stents. More particularly, the present invention relates to intraluminal support devices that include a graft material. Also, the present invention relates to delivery and placement systems for deploying an intraluminal support device in a particular area within a body vessel.

### BACKGROUND OF THE INVENTION

Various types of disease conditions present clinical situations in which a vessel of a patient needs to be artificially supported or held in an open condition. For example, blood flow through an artery can be impeded due to a build-up of cholesterol. Also, the wall of a vessel may be weakened by an aneurysm.

Intraluminal support frames, sometimes referred to as stents, provide an artificial mechanism to support a body vessel. These support frames can be fabricated from various materials, such as metals and plastics, and fall broadly into two categories: self-expanding and balloon-expandable support frames.

Self-expanding support frames are able to take on a radially compressed configuration, which facilitates delivery of the frame to the site of interest. Once at the site, a constraining force holding the frame in the radially compressed configuration is removed, and the frame automatically takes on a radially expanded configuration. In this configuration, the frame exerts a radially outward force on the vessel, which supports the vessel.

Other support frames require the application of, as opposed to the removal of, an external force to expand the frame into the supportive configuration. Typically, an inflatable balloon is used to radially expand these support frames. The balloon is commonly placed on the tip of a delivery catheter, inside the support frame. Upon inflation, the underlying balloon forces the overlying support frame to radially expand, which allows the frame to support the vessel.

Typically, both types of support frames are formed in a mesh-like pattern or other open weave pattern. While such patterns allow for radial expansion, they also concentrate the outward force exerted on the vessel by the frame to the individual threads within the mesh. Little or no force is exerted on the vessel wall in the open cells or empty spaces of the mesh structure. As a result, tissue in-growth can occur in these spaces, which may lead to restenosis of the vessel and may necessitate additional treatment for the patient. Ultimately this condition may require replacement of the support frame.

To address the tissue ingrowth problem, several devices have been proposed that combine a graft material with a support frame. The use of a graft provides a continuous surface for supporting the vessel and works to minimize the penetration of tissue into the open cells of the support frame. Most prior art devices include a graft that completely surrounds the support frame. While these devices may lessen the ingrowth of tissue, this approach may limit the ability of the support frame to radially expand because the surrounding graft places a constraining force on the frame. Such a limitation of expansion may also impede the flexibility of the support frame once deployed, which could render the device less effective.

Other prior devices utilize a graft disposed on a portion of a support frame with a variable circumference. For example, U.S. Pat. No. 6,231,597 to Deem et al. discloses a support frame with two different regions—a partial circumference region and a full circumference region. The partial circumference region partially engages the inner surface of the wall of a vessel, while the other region fully engages the wall. A graft can be placed on one of these regions. Unfortunately, the use of a partial circumference region in this device leaves a portion of a treated vessel without support.

In view of these and other deficiencies of the prior art, there is a need for an intraluminal support device that has a substantially uniform circumference and a graft material disposed about a portion of the circumference.

### SUMMARY OF THE INVENTION

The present invention provides an intraluminal support device for providing support to a body vessel, such as an artery. In one embodiment, the support device comprises a support frame having a tubular structure formed of one or more frame threads that define a plurality of open cells. The support frame has a substantially uniform circumference along its length. A graft material is disposed on a portion of the support frame. The graft material spans one or more cells along the length of the support frame and extends only a partial distance along the circumference of the support frame.

Various types of support frames can be used in the support device of the present invention, including both self-expanding and balloon expandable frames. For example, a mesh like structure formed from a single frame thread can be used. Also, a pattern formed in a sheet of biocompatible material can be utilized. The specific type of support frame used will depend upon the ultimate application of the support device.

The present invention also provides an apparatus for delivering an intraluminal support device to a vessel. In one embodiment, a delivery apparatus according to the present invention comprises a first catheter having a distal end and an intraluminal support device disposed on the distal end. The support device has characteristics according to the present invention. A balloon is positioned on the distal end of the first catheter, inside the support device. The balloon is adapted to circumferentially expand the support frame of the intraluminal support device upon inflation of the balloon. The delivery apparatus can further include a second retractable catheter that surrounds the first catheter and the intraluminal support device. In use, this second catheter is retracted to allow circumferential expansion of the support frame by the balloon.

While the invention is defined by the appended claims, additional understanding of the invention can be achieved by referencing the following figures and detailed description of preferred embodiments of the invention.

### BRIEF DESCRIPTION OF THE DRAWINGS

FIG. 1 is a perspective view of an intraluminal support device according to a first preferred embodiment of the invention.

FIG. 2 is a cross-sectional view taken along line 2-2 in FIG. 1.

FIG. 3 is a perspective view of a first alternate embodiment of the support device illustrated in FIG. 1.

FIG. 4 is a perspective view of a second alternate embodiment of the support device illustrated in FIG. 1.

FIG. 5 is a perspective view, partially broken away, of an intraluminal support device according to a second preferred embodiment of the invention.

FIG. 6 is a plan view of the support device of FIG. 5 in a flat configuration.

FIG. 7 illustrates a perspective view, broken away, of an intraluminal support device according to a third preferred embodiment of the present invention.

FIGS. 8A and 8B are schematic representations of fluoroscopy images of the device illustrated in FIG. 7 placed within a body vessel.

FIG. 9 is a partial cross-sectional view, partially broken away, of a delivery apparatus according to a preferred embodiment of the invention.

FIG. 10 is a schematic of the use of an intraluminal support device according to the present invention to exclude an aneurysm from the circulation in a body vessel.

### DETAILED DESCRIPTION OF PREFERRED EMBODIMENTS OF THE INVENTION

The following description of preferred embodiments of the invention provides examples of the present invention. The embodiments discussed herein are merely exemplary in nature, and are not intended to limit the scope of the invention in any manner. Rather, the description of these preferred embodiments serves to enable a person of ordinary skill in the relevant art to make and use the present invention.

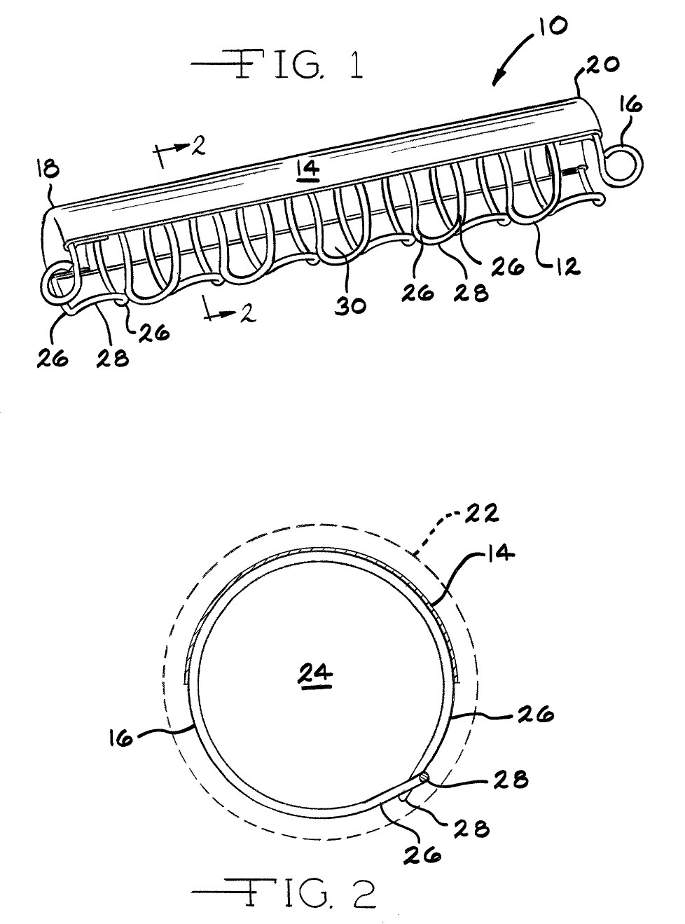

FIGS. 1 and 2 illustrate an intraluminal support device according to a first preferred embodiment of the present invention. The device 10 comprises a support frame 12 and graft 14.

The support frame 12 is formed from one or more frame threads 16 that are wound into a tubular structure. As best illustrated in FIG. 1, the threads 16 are preferably wire like structures formed of the material chosen for the support frame 12. The threads 16 can be selected from a wide variety of materials. The material chosen should allow the support frame to expand circumferentially (i.e., radially) and should also be medically acceptable (e.g., biocompatible). Accordingly, the frame threads 16 can be formed of a wide variety of natural and synthetic materials including collagen, various thermoplastics, and various metals. Examples of suitable thermoplastics include polyester, polypropylene, polyethylene, polyurethane, polytetrafluroethylene (PTFEs), and combinations and mixtures thereof. Examples of suitable metals include stainless steel, titanium, nickel chromium alloys, and nickel titanium alloys. Preferred materials include stainless steel and nickel titanium alloys known to those skilled in the art.

The tubular structure formed by the frame threads 16 has a first end 18, a second end 20, and a substantially uniform circumference along its length (i.e., from the first end 18 to the second end 20). As best illustrated in FIG. 2, the uniform circumference 22 preferably comprises a full circle. This allows the intraluminal support device 10 to engage the entire interior circumference of a particular cross-section of a vessel into which the device 10 is introduced. Alternatively, any type of partially circular circumference could be employed. For example, a C-shaped circumference can be utilized. No matter the circumference chosen, the circumference is substantially uniform along the entire length of the device. That is, the circumference remains substantially the same along the length from the first end 18 to the second 20 of the device 10.

As illustrated in FIGS. 1 and 2, the tubular structure is preferably formed by a single frame thread 16. The frame thread 16 can be woven together in any suitable pattern. The pattern illustrated in the figures merely represents a preferred pattern for use in the present invention. A wide variety of industrial weaving patterns and techniques are known to those skilled in the art, and any suitable pattern can be used. The pattern chosen need only provide the uniform circumference along the length of the support device 10.

Preferably, as best illustrated in FIG. 1, a single frame thread 16 is wound to form a plurality of ring segments 26 connected by a plurality of curved regions 28. Particularly preferable, as best illustrated in FIG. 2, adjacent curved regions 28 extend beyond each other such that adjacent ring segments 26 are interleaved. This particular pattern provides the preferred full circle circumference 22 and confers a degree of radial flexibility onto the device 10.

The one or more frame threads 16 define a plurality of open cells 30. The size, shape, and configuration of the cells 30 will depend on various factors, such as the type of thread and the weaving pattern utilized. As illustrated in FIG. 1, the cells 30 can be formed by a simple weaving pattern in a single frame thread 16. Alternatively, cells can be formed by intersections of single or multiple frame threads. Also, the cells do not have to be completely bound by a frame thread—an open area may exist. In all embodiments, the cells 30 comprise an open space between portions of the one or more frame threads.

The graft material 14 can comprise any suitable graft material known in the art. Examples of acceptable material include mesh material, woven materials, such as fabric and Dacron (Dacron is a registered trademark of the E. I. DuPont DeNemours Company), and synthetics such as polypropylene. Also, natural materials such as collagen and extracellular matrix (ECM) materials can be used. A preferred graft material is small intestine submucosa (SIS), such as SIS harvested from swine. The preparation and use of SIS, in contexts other than that of the present invention, are known to those skilled in the art. Descriptions of this material and procedures for its preparation can be found in U.S. Pat. No. 4,902,508 to Badylak et al. for a TISSUE GRAFT COMPOSITION, which is hereby incorporated into this disclosure in its entirety.

As shown in FIGS. 1 and 2, the graft material 14 is disposed on a portion of the support frame 12 and preferably spans one or more of the cells 30 formed by the one or more frame threads 16. Particularly preferable, as best illustrated in FIG. 1, the graft material 14 extends along the entire length of the support frame 12, i.e., from the first end 18 to the second end 20, and spans a portion of each cell 30 formed by the frame threads 16. Also, the graft material 14 extends only a partial distance along the circumference 22 of the support frame 12. Thus, the graft material 14 does not extend along the entire circumference 22 of the support frame of the device 10.

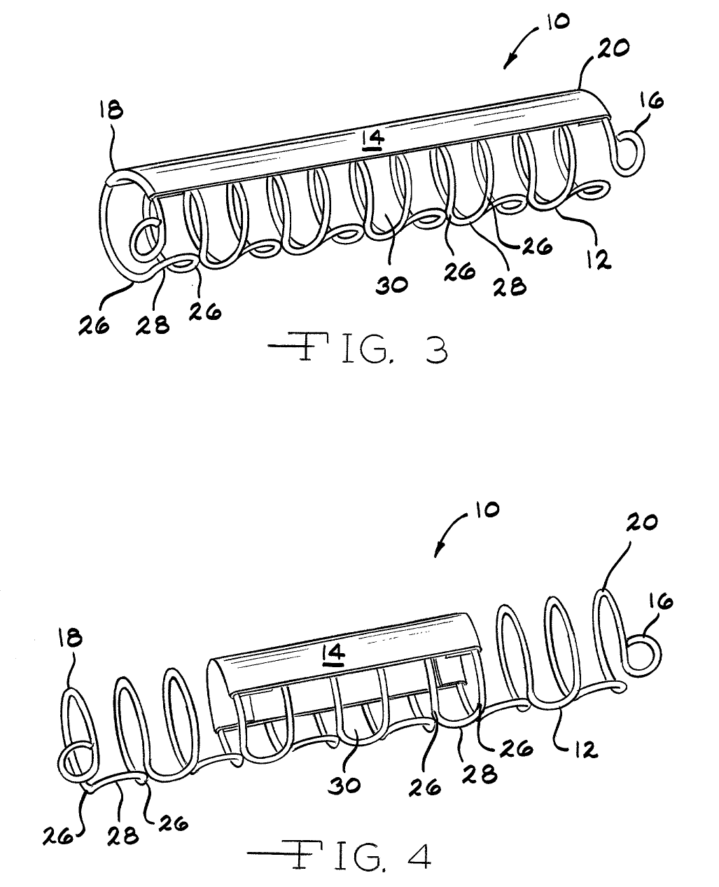

The partial distance along which the graft material 14 extends can be any suitable partial distance. The actual partial distance utilized will depend upon many factors, including the size of vessel to be treated and the relative size of the lesion or other type of treatment site within the vessel. As illustrated in FIGS. 1 and 2, the partial distance preferably comprises approximately ½ of the entire circumference 22 of the tubular structure. FIG. 3 illustrates an alternative of this preferred embodiment in which the partial distance comprises approximately ¼ of the circumference 22.

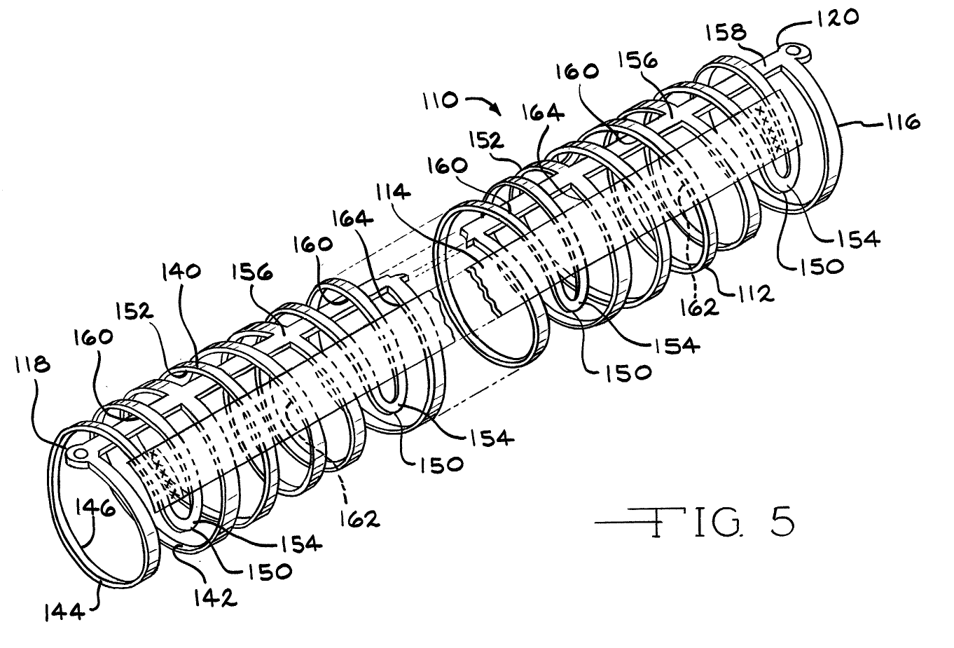

The graft material 14 preferably extends along the entire length of the intraluminal support device 10 (i.e., from the first end 18 to the second end 20). As illustrated in FIG. 4, the graft material 14 may, however, extend along a fractional length of the device 10. Again, as with the partial distance of the circumference discussed above, the actual fractional length utilized will depend on several factors, including the size of the lesion and/or weakened area of the vessel being treated. Furthermore, the graft material 14 can be situated near either end of the device 10, leaving the support frame 12 exposed at the opposite end. Alternatively, as illustrated in FIG. 4, the graft material 14 can be centered, leaving the support frame 12 exposed at both the first 18 and second 20 ends.

Of course, the various possible fractional lengths can be combined with the various possible partial distances along the circumference to create a wide array of stent products useful in treating a variety of clinical situations. For example, if a vessel contains a relatively short lesion along approximately ½ of its circumference, an intraluminal support device according to the present invention may include a fractional length graft disposed at one end of the device and extending along approximately ½ of the circumference of the device.

The graft material 14 can be secured to the support frame 12 in a variety of ways. For example, the graft can be formed over a portion of the frame 12, effectively embedding the frame 12 in the graft material 14. This arrangement is suitable for graft materials 14 that can be formed around other materials, and is thus acceptable for polymeric graft materials. Alternatively, the graft material 14 can be secured to the support frame 12 by suitable attachment means. This arrangement is particularly advantageous for graft materials 14 formed into sheet configurations, such as synthetics and ECMs. Preferably, the graft material 14 in this arrangement simply lies on the support frame 12, covering the portion of the frame 12 that contacts the graft 14. Various attachment means can be employed to secure the graft 14 in this manner. For example, as best illustrated in FIG. 1, the ends of the graft material 14 can be folded around a section of a frame thread 16 and back onto itself, creating an area of double thickness. An adhesive or other suitable securement means, such as sutures, can then be used in the double thickness area to connect the two layers of the graft material 14, effectively securing the graft material 14 to the support frame 12.

FIG. 5 illustrates a second preferred embodiment of the present invention. This embodiment is similar to the previous preferred embodiment, except as indicated below. Accordingly, references in FIG. 5 are a 100 series of numbers and like references refer to similar features and/or components illustrated in FIGS. 1 through 4 and discussed above.

In this embodiment, the support frame 112 includes a frame thread 116 that comprises a pattern formed from a seamless sheet of a biocompatible material, such as stainless steel. The pattern can be formed from the sheet by various methods known to those skilled in the art, such as photo etching. An example of such a support frame, without a graft material in accordance with the present invention, is provided in U.S. Pat. No. 5,632,771 to Boatman et al. for a FLEXIBLE STENT HAVING A PATTERN FROM A SHEET OF MATERIAL. These frames provide various advantages over conventional wire support frames, such as the elimination of weaving steps during the manufacturing process, an integral structure, and the provision of various flat surfaces throughout the frame.

As illustrated in FIG. 5, the frame thread 116 in this embodiment preferably has flat surfaces 140, 142, 144, and 146. The availability of these various flat surfaces facilitates attachment of the graft material 114 to the support frame 112. Preferably, as illustrated in FIG. 5, the graft material 114 is folded around the flat surfaces 140, 142, 144, and 146 and doubled back upon itself. Sutures 148 are used to secure the graft material 114 to itself, and effectively to the support frame 112.

As illustrated in FIG. 5, the support frame 112 preferably defines two series 150, 152 of opposing fingers. Each finger 150, 152 includes a distal end 154 extending away from a base 156 integrally formed with a longitudinal support 158 of the support frame 112. Each finger 150, 152 is circumferentially wrapped around the longitudinal support 158 to define the circumference 122 of the device 110. The distal end 154 of each finger 150, 152 overlaps the longitudinal support 158, providing an increased height 160 at the point of overlap. The base 156 of each finger 150, 152 is integral with the longitudinal support 158, so no increased height occurs at the juncture between the base 156 and the longitudinal support 158.

As in the first preferred embodiment, the graft material can be secured to the support frame 112 along any partial distance of the circumference 122. As illustrated in FIG. 5, the graft material 114 is preferably secured to the distal ends 154 of one series of fingers 150. Due to the increased height 160, the graft material 114 rests against a curved region 162 of each finger 152 in the opposite series of fingers 152. As a result, a clearance 164 exists at the bases 156 of the fingers 152 of the first series because the increased height 160 is absent in this area. This arrangement is advantageous as it provides flexibility to the surface of the graft material 114, which can allow a vessel to better accommodate the support device 110.

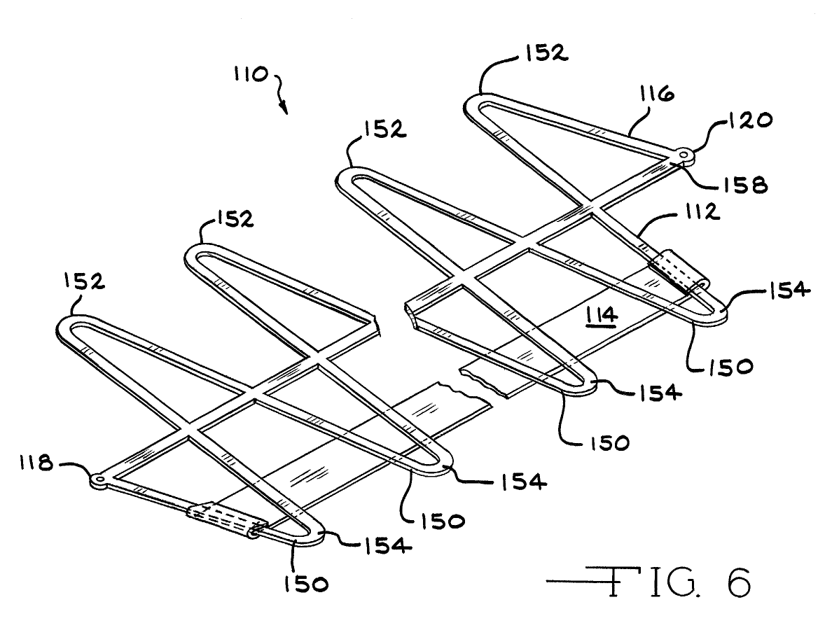

The intraluminal support device of the present invention can be fabricated by attaching a graft material to a pre-formed support frame. For example, a graft material can be molded onto a wire frame or can be secured to a frame with various attachment means, such as sutures. If the support frame comprises a pattern formed from a sheet of material, as in the second preferred embodiment, the graft material is preferably secured to the support frame 12 while in the flat, i.e., sheet, configuration. FIG. 6 illustrates the device 110 of FIG. 5 in its sheet configuration. In this Figure, the support frame 112 has been cut from a sheet of biocompatible material and lies in a flat configuration. As illustrated in the Figure, the graft material 114 is attached to the fingers 150 of the frame 112 prior to rolling the frame 112 into its final, tubular configuration. After attaching the graft material 114 to this flat support frame 112, the frame 112 is formed into the tubular structure by any suitable process, such as by rolling or curling the opposing fingers 150, 152. During this fabrication process, the fingers 152 not attached to the graft 114 are passed under the graft 114, preferably in a manner that allows the graft 114 to rest against these fingers 152.

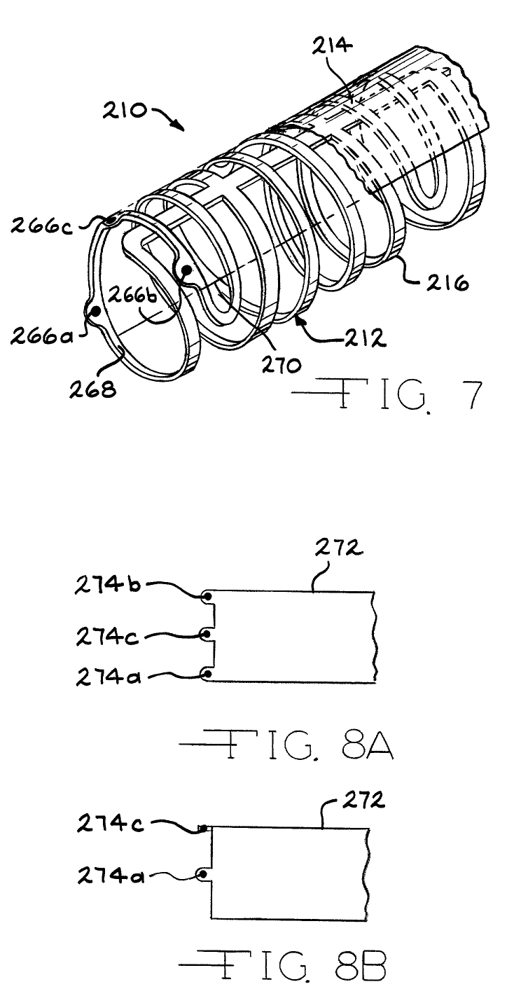

FIG. 7 illustrates a portion of an intraluminal support device according to a third preferred embodiment of the present invention. This embodiment is similar to the second preferred embodiment, except as indicated below. Accordingly, references in FIG. 7 are a 200 series of numbers, and like references refer to similar features and/or components illustrated in FIGS. 5 and 6.

In this embodiment, the graft 214 extends along approximately ½ the circumference of the support frame 212. Also, the device 210 includes radiopaque markers 266 to aid in the positioning of the device 210 within a vessel. The radiopaque markers 266 can comprise any suitable marker known to those skilled in the art. Preferably, as illustrated in FIG. 7, each marker comprises a rivet that passes through the frame thread 216. Preferably gold rivets are utilized.

To facilitate orientation of the device 210 in a vessel relative to the graft 214, the radiopaque markers 266 are preferably positioned at positions on the frame thread 216 that correspond, in some manner, with the graft material 214. In a particular preferred embodiment, one marker 266a, 266b is positioned on the frame thread 216 at a point adjacent the lateral edge 268, 270 of the graft 214. Furthermore, a third marker 266c is positioned at some point between the end markers 266a, 266b and adjacent the graft material 214.

FIGS. 8A and 8B illustrate the use of the markers to position the device illustrated in FIG. 7 in a vessel 272 relative to the graft material 214.

FIG. 8A shows a schematic conventional fluoroscopy image of the vessel 272 containing the device. In this image, three spots 274a-c appear, corresponding to the three markers 266a-c. Since all three markers 266a-c are represented, the graft is either toward or away from the viewer. However, if only two spots 274a, 274c appear, as in the image illustrated in FIG. 8B, the device is positioned in the vessel 274 with the graft material on a particular side of the vessel 272. This can be used to position the graft material on the side of a vessel in need of treatment, as described below.

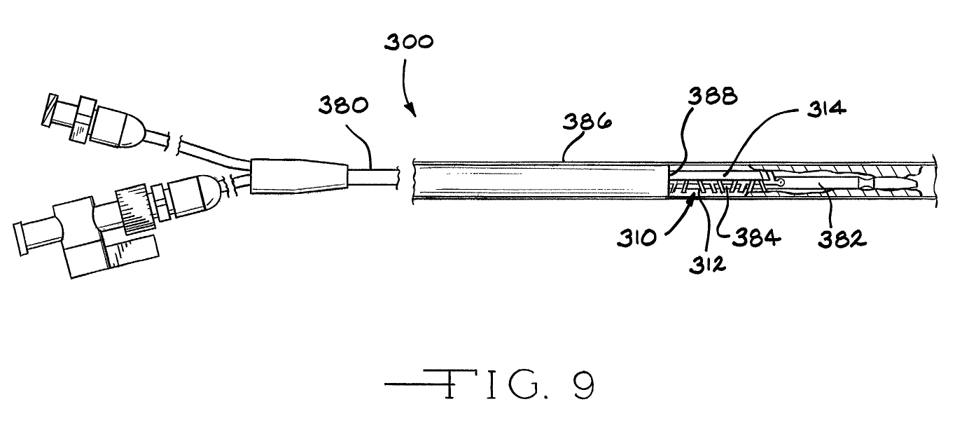

The present invention also provides an apparatus for delivering an intraluminal support to a site in a vessel that is in need of artificial support. As discussed below, the delivery apparatus according to the present invention includes an intraluminal support device in accordance with the invention. FIG. 9 illustrates a preferred embodiment of a delivery apparatus 300 according to the present invention. Thus, references in FIG. 9 are a 300 series of numbers, and like references refer to similar features and components illustrated in the previous figures.

In one embodiment, the delivery apparatus 300 includes a first catheter 380 having a distal end 382. A support frame 312 of an intraluminal support device 310 according to the present invention is disposed on the distal end 382. The support device 310 is formed into a tubular structure as described above and surrounds the distal end 382 of the first catheter 380. A graft material 314 is disposed on the support frame 312 in accordance with the present invention as discussed above. Also, an inflatable balloon 384 is attached to the distal end 382 of the first catheter 380 and positioned inside the support frame 312 of the intraluminal support device 310. The balloon 384 is selectively inflatable and adapted to circumferentially expand the support frame 312 in the conventional manner.

The delivery apparatus 300 may also include a second catheter 386 that defines a lumen 388. The second catheter 386 provides protection to the support frame 312 during navigation through body vessels. Also, the second catheter 386 shields the vessel wall from the surface of the support frame 312 during navigation.

In this embodiment, the first catheter 380 and the attached intraluminal support device 310 and balloon 384 are disposed within the lumen 388 of the second catheter 386. When the second catheter 386 surrounds the distal end 382 of the first catheter 380, the balloon 384 cannot expand the support frame 312 due to the presence of the second catheter 386. Upon retraction, the second catheter 386 is withdrawn from a position surrounding the support device 310. Accordingly, following retraction of the second catheter 386, the support device 310 is exposed and the balloon 384, upon inflation, is able to circumferentially expand the support frame 312 of the intraluminal support device 310. This circumferential expansion allows the deployment of the support device 310 at the point in the vessel in need of artificial support.

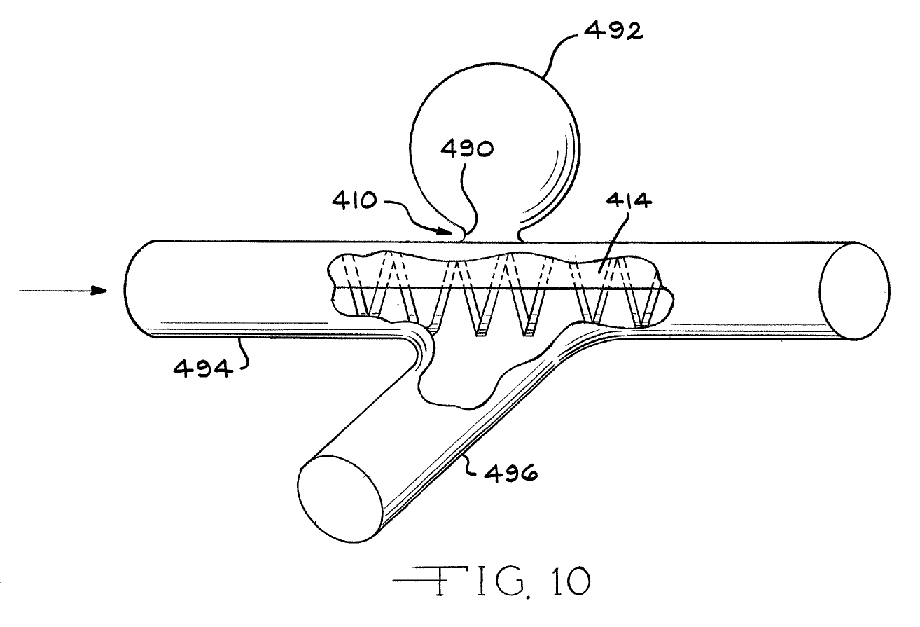

The intraluminal support device and delivery apparatus of the present invention are particularly well suited for treatment to exclude aneurysms from a body vessel while still allowing flow through the vessel. FIG. 10 illustrates a schematic of the use of the present invention to exclude an arterial aneurysm. As shown in the figure, the intraluminal support device 410 is positioned such that the graft material 414 blocks the neck or opening 490 of the aneurismal sack 492. This blocks blood flow to and relieves pressure from the sack 492, thereby reducing the danger of an aneurismal rupture. The graft 414 can be positioned to block the opening 490 using radiopaque markers as described above. The side of the device 410 that does not include the graft 414 allows circulation to flow through the non-blocked or non-excluded portion of the vessel. This is particularly advantageous when the aneurismal sack 492 is positioned at a junction between a main artery 494 and a branch artery 496.

In this treatment approach, the support device of the present invention provides immediate blockage to the sack while protecting the opening and vessel at the point of aneurysm from the edges of an uncovered support device, thereby lowering the stress typically associated with conventional treatments, such as percutaneous or trans-catheter methods.

All references cited in this disclosure are hereby incorporated into this disclosure in their entirety, except to any extent to which they contradict any definition or statement contained herein.

The foregoing disclosure includes the best mode devised by the inventor for practicing the invention. It is apparent, however, that several variations in accordance with the present invention may be conceivable by one skilled in the art. Inasmuch as the foregoing disclosure is intended to enable one skilled in the pertinent art to practice the instant invention, it should not be construed to be limited thereby, but should be construed to include such aforementioned variations. As such, the present invention should be limited only by the spirit and scope of the following claims.

I claim:

1. An intraluminal support device for providing support to a body vessel, comprising:
a support frame comprising a tubular structure formed of one or more frame threads and having a length and a substantially uniform circumference; and
a graft material disposed on a portion of the support frame and spanning at least a portion of the length, the graft material extending only a partial distance along the circumference of the support frame.

2. The intraluminal support device of claim 1, wherein the one or more frame threads define a plurality of cells; and
wherein the graft material spans at least one of the plurality of cells.

3. The intraluminal support device of claim 1, wherein the uniform circumference is adapted to engage the entire interior circumference of a particular cross-section of said vessel.

4. The intraluminal support device of claim 1, wherein a single frame thread forms the support frame.

5. The intraluminal support device of claim 4, wherein the frame thread defines a plurality of ring segments joined together by curved regions.

6. The intraluminal support device of claim 5, wherein adjacent curved regions extend beyond each other such that adjacent ring segments are interleaved.

7. The intraluminal support device of claim 4, wherein the frame thread comprises a pattern formed from a sheet of biocompatible material.

8. The intraluminal support device of claim 1, wherein the graft material extends from a first end to a second end of the support frame.

9. The intraluminal support device of claim 1, wherein the graft material extends along a fractional length of the length of the support frame.

10. The intraluminal support device of claim 1, wherein the portion of the support frame contacting the graft material is embedded in the graft material.

11. The intraluminal support device of claim 1, wherein the graft material covers the portion of the support frame contacting the graft material.

12. The intraluminal support device of claim 11, further comprising attachment means for connecting the graft material to the support frame.

13. The intraluminal support device of claim 1, wherein the partial distance along the circumference of the support frame is approximately ½ of the circumference.

14. The intraluminal support device of claim 1, wherein the partial distance along the circumference of the support frame is approximately ¼ of the circumference.

15. The intraluminal support device of claim 1, further comprising at least one radiopaque marker disposed on a frame thread.

16. The intraluminal support device of claim 15, wherein the graft material has first and second lateral edges along the circumference of the support frame;
wherein the at least one radiopaque marker comprises first and second radiopaque markers;
wherein the first radiopaque marker is positioned adjacent the first lateral edge; and
wherein the second radiopaque marker is positioned adjacent the second lateral edge.

17. The intraluminal support device of claim 16, wherein the at least one radiopaque marker further comprises a third radiopaque marker disposed between the first and second radiopaque markers and adjacent the graft material.

18. An intraluminal support device, comprising:
a frame thread formed into a plurality of interleaved ring segments joined together by curved regions to form a tubular structure having a first end, a second end, and defining a plurality of cells that provide a substantially uniform circumference to the tubular structure; and
a graft material extending only a partial distance along the circumference of the tubular structure and from the first end to the second end such that the graft material spans a portion of each cell of the plurality of cells.

19. The intraluminal support device of claim 18, wherein the partial distance along the circumference of the tubular structure is approximately ½ of the circumference.

20. The intraluminal support device of claim 18, wherein the partial distance along the circumference of the tubular structure is approximately ¼ of the circumference.

21. An intraluminal support device, comprising:
a support frame formed from a pattern in a sheet of biocompatible material, the support frame comprising a tubular structure having a first end, a second end, and defining a plurality of cells that provide a substantially uniform circumference to the support frame; and
a graft material extending only a partial distance along the circumference of the support frame and from the first end to the second end such that the graft material spans a portion of each cell of the plurality of cells.

22. The intraluminal support device of claim 21, wherein the partial distance along the circumference of the support frame is approximately ½ of the circumference.

23. The intraluminal support device of claim 21, wherein the partial distance along the circumference of the support frame is approximately ¼ of the circumference.

24. The intraluminal support device of claim 21, further comprising attachment means for connecting the graft material to the support frame.

25. The intraluminal support device of claim 21, further comprising at least one radiopaque marker disposed on the support frame.

26. The intraluminal support device of claim 25, wherein the graft material has first and second lateral edges along the circumference of the support frame;
wherein the at least one radiopaque marker comprises first and second radiopaque markers;
wherein the first radiopaque marker is positioned adjacent the first lateral edge; and
wherein the second radiopaque marker is positioned adjacent the second lateral edge.

27. The intraluminal support device of claim 26, wherein the at least one radiopaque marker further comprises a third radiopaque marker disposed between the first and second radiopaque markers and adjacent the graft material.

28. An apparatus for delivering an intraluminal support device to a vessel, comprising:
a first catheter having a first distal end;
a balloon positioned on the first distal end;
a support frame surrounding the balloon and comprising a tubular structure formed of one or more frame threads defining a plurality of cells that provide a substantially uniform circumference to the support frame; and
a graft material disposed on a portion of the support frame and spanning at least a portion of one or more cells of the plurality of cells, the graft material extending only a partial distance along the circumference of the support frame;
wherein the balloon is adapted to circumferentially expand the support frame upon inflation.

29. The apparatus of claim 28, further comprising a retractable second catheter having a second distal end defining a lumen;
wherein the support frame and first catheter are disposed in the lumen of the second distal end.

30. An intraluminal support device, comprising:
a support frame comprising a tubular structure having a uniform circumference and formed of a pattern in a sheet of biocompatible material, the pattern defining first and second series of opposing fingers connected by a longitudinal support; and
a graft material attached to first and second fingers of the first series of opposing fingers and extending only a partial distance along the circumference of the support frame.

31. The intraluminal support device of claim 30, wherein a portion of at least one finger of the second series of fingers lies below the graft material.

32. The intraluminal support device of claim 31, wherein a clearance exists between a portion of the first and second fingers of the first series of fingers and the graft material.

33. The intraluminal support device of claim 30, wherein the graft material is attached to distal ends of the first and second fingers.

34. The intraluminal support device of claim 30, wherein the partial distance along the circumference of the support frame is approximately ½ of the circumference.

35. The intraluminal support device of claim 30, wherein the partial distance along the circumference of the support frame is approximately ¼ of the circumference.

36. The intraluminal support device of claim 30, further comprising at least one radiopaque marker disposed on the support frame.

37. The intraluminal support device of claim 36, wherein the graft material has first and second lateral edges along the circumference of the support frame;
wherein the at least one radiopaque marker comprises first and second radiopaque markers;
wherein the first radiopaque marker is positioned adjacent the first lateral edge; and
wherein the second radiopaque marker is positioned adjacent the second lateral edge.

38. The intraluminal support device of claim 37, wherein the at least one radiopaque marker further comprises a third radiopaque marker disposed between the first and second radiopaque markers and adjacent the graft material.
